(19) United States
(12) Patent Application Publication
(10) Pub. No.: US 2008/0096970 A1
(43) Pub. Date: Apr. 24, 2008

(54) Stable Nateglinide Form B Compositions

(76) Inventors: Sundaram Venkataraman, Hyderabad (IN); Sharat Pandurang Narsapur, Secunderabad (IN); Manoj Ramesh Kharkar, Hyderabad (IN); Rongali Bangarubabu, Vizianagaram (Dist) (IN); Mohanty Sandeep, Orissa (IN); Pyne Sayantani, Kolkata (IN); Kakartapudi Ranga Raju, Visakhapatnam Dt. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/569,382

(22) PCT Filed: May 20, 2005
(86) PCT No.: PCT/US05/17664

§ 371 Date: Nov. 20, 2006

Related U.S. Application Data
(60) Provisional application No. 60/572,689, filed on May 20, 2004.
(60) Provisional application No. 60/586,431, filed on Jul. 8, 2004.
(60) Provisional application No. 60/644,614, filed on Jan. 18, 2005.

(51) Int. Cl. A61K 31/165; C07C 233/00

(52) U.S. Cl. 514/617; 564/161

(57) ABSTRACT

A process for preparing nateglinide Form B comprises dissolving nateglinide in a solvent and adding the solution, at temperatures of 40-45° C., to a hydrocarbon liquid that is at temperatures of 40-45° C. Then, water is added and the mixture is allowed to cool, producing crystals of nateglinide Form B.

### INTRODUCTION TO THE INVENTION

The present invention relates to compositions containing stable nateglinide Form B crystals.

Nateglinide is chemically known as (−)-N-[(trans-4-isopropylcyclohexane)-carbonyl]-D-phenylalanine and is represented by the following Formula I:

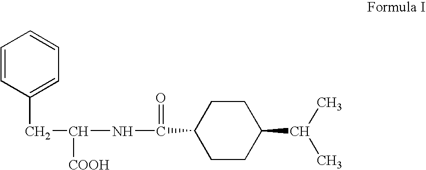

Nateglinide is a substance having therapeutic utility in depressing blood glucose levels. It shows hypoglycemic action, by stimulating the secretion of insulin from the pancreas when plasma glucose concentrations are elevated. A commercial nateglinide product is being sold using the tradename STARLIX.

The compound nateglinide is known to exist in different crystalline forms. U.S. Pat. Nos. 5,463,116 and 5,488,150 relate to a polymorphic form known as “Form H.” U.S. Patent Application 2003/0229249 A1 relates to the polymorphic form known as “Form B.”

It is very well known from U.S. Pat. No. 5,463,116 and other literature that nateglinde Form B crystals have problems of instability, especially when subjected to mechanical operations such as size reduction. The instability of the Form B crystals means that they are not ideal for use in medicine. It is generally desirable that a medicinal product contains an active ingredient having a composition which is well defined and predictable in terms of properties, including the crystallinity of the active ingredient.

Accordingly, it is desirable to obtain nateglinide Form B that will not change its polymorphic form during procedures that are required to formulate a pharmaceutical dosage form.

### SUMMARY OF THE INVENTION

The present invention relates to methods for preparing stable nateglinide Form B crystals. By use of the invention, nateglinide Form B crystals can be used to prepare fine particle sizes that are suitable for formulating pharmaceutical dosage forms.

In one aspect, the invention comprises preparing crystals of nateglinide Form B that are resistant to polymorphic form conversion during formulation processing procedures, by dissolving nateglinide in a solvent, adding the solution to a hydrocarbon liquid, adding water to the mixture, and isolating formed nateglinide Form B crystals.

In another aspect, the present invention comprises a process for preparing a mixture of nateglinide Form B crystals and at least one pharmaceutical excipient, which mixture can subsequently be subjected to particle size reduction procedures without changing the polymorphic form of the nateglinide. The process comprises mixing excipients with a chilled organic liquid, adding nateglinide Form B crystals, mixing, and then separating solids from the liquid.

### BRIEF DESCRIPTION OF THE DRAWINGS

FIG. 1 is an X-ray diffraction pattern of nateglinide Form B crystals, prepared according to the present invention.

FIG. 2 is a differential scanning calorimetry thermogram of nateglinide Form B crystals, prepared according to the present invention.

FIG. 3 is an X-ray diffraction pattern of a nateglinide-excipient mixture prepared according to the present invention.

FIG. 4 is a particle size distribution graph of a nateglinide-excipient mixture prepared according to the present invention.

### DETAILED DESCRIPTION

One aspect of the present invention relates to the preparation of nategliniude Form B crystals that are resistant to polymorphic changes during processing into pharmaceutical dosage forms, such processing including steps such as particle size reduction. The nateglinide Form B crystals are prepared by dissolving nateglinide in a solvent and adding the solution, at temperatures of 40-45° C., to a hydrocarbon liquid that is at temperatures of 40-45° C. Then, water is added and the mixture is allowed to cool, producing crystals of nateglinide Form B. After removing the liquid, the nateglinide Form B can be used to produce a pharmaceutical dosage form, or can be further stabilized as a mixture with an excipient using the aspect of the invention described below.

Optionally, the solution of nateglinide in the solvent can be treated with a decolorizing agent such as carbon, then filtered to remove the carbon and any undissolved nateglinide.

Nateglinide that is useful as a starting material for the process can be any form of the compound, including Form B that has been prepared by this or any other process.

The solvent for nateglinide can be an alcohol having 1 to about 4 carbon atoms, either branched or unbranched, or a ketone formed by one or two alkyl groups independently having 1 to about 4 carbon atoms, either branched or unbranched. The hydrocarbon liquid generally will have about 4 to about 8 carbon atoms, and be either branched or unbranched.

In general, the amount of solvent that is used to dissolve the nateglinide should be only slightly in excess of the minimum quantity required, to maximize the final recovery of nateglinide Form B. Hydrocarbon liquid will generally be used in an amount that is between about 5 and about 10 times the volume of solvent for the nateglinide. The quantity of water used generally will be about 2 to about 4 times the volume of solvent. These are merely guidelines, as using excess quantities of hydrocarbon liquid and/or water will not substantially affect the process outcome but will cause some inconvenience for the operator and affect process economics, environmental impact, and the like.

As an example showing useful proportions, 2.5 kg of nateglinide can be dissolved in 5 L of isopropanol, and the solution added to 37.5 L of n-heptane under appropriate temperature conditions. Then, 15 L of water can be added and crystals of nateglinide Form B recovered. Other quantities can be used, as discussed above.

Another aspect of the present invention relates to a process for the preparation of a mixture of nateglinide Form B crystals with a pharmaceutical excipient, in which mixture the nateglinide will be stable against polymorphic change during subsequent particle size reduction procedures.

A representative process for the preparation of the mixture comprises the following steps:

a. chilling an organic liquid to temperatures below ambient, then adding one or more pharmaceutical excipients and mixing while maintaining the low temperatures;

b. adding nateglinide Form B crystals and continuing the mixing; and

c. separating a solid, which is a mixture of nateglinide Form B and the excipient or excipients.

Low temperatures, such as less than about 10° C., are particularly useful for chilling the organic liquid before excipient is added.

The starting nateglinide Form B for this process can be prepared using the procedure described above, or can be any other nateglinide having polymorphic Form B.

The quantity of organic liquid can be selected for the convenience of the operator. Usually, that amount will be the minimum sufficient to provide a desired fluidity to the final slurry, thereby providing required handling properties. The use of excess quantities of organic liquid should be avoided to minimize the expense of the process and for protection of the environment.

In general, the amount of solid excipient or excipient mixture used can be from about 0.2 to about 5 times the amount of the nateglinide, on a weight basis. It sometimes is preferred for the excipient or excipient mixture to be about equal in weight to the nateglinide.

Useful excipients for this embodiment of the invention include: starches, or starch derivatives such as sodium starch glycolate; sugars such as lactose, mannitol, sucrose, glucose, fructose, or aldose; microcrystalline cellulose or other cellulose forms and derivatives; alkaline earth metal salts, such as phosphates; aluminosilicates such as bentonite or kaolin; inorganic oxides; or mixtures of any two or more substances from these categories. In general, the excipient should be chosen to be insoluble, or only slightly soluble, in the organic liquid. The foregoing listing is not intended to be exhaustive, but merely representative of the many substances that can be used.

Useful organic liquids include hydrocarbons such as: aliphatic alkanes having up to about ten carbon atoms in a straight or branched configuration, such as ethane, propane, butane, pentane, hexane, heptane, or mixtures thereof; hydrocarbon mixtures such as petroleum ether; and ethers, such as isopropyl ether, t-butyl ether, and the like. The organic liquid should not be a solvent in which nateglinide has an appreciable solubility.

After separation of the solid mixture of nateglinide Form B and the excipient or excipients, drying will typically be desired. This drying can be accomplished by any known method, such as air tray drying, use of a rotational dryer such as the Buchi Rotovapor, vacuum tray drying, microwave oven drying, fluidized bed drying, flash drying, spin flash drying, and others.

Stability of the nateglinide Form B after processing is determined by an absence of the polymorphic Form H of the compound. This is determined by generating X-ray powder diffraction patterns of samples and checking for the characteristic peaks that correspond to Form H. For the present application, the X-ray diffraction patterns of the figures were obtained with Cu K_{α-1} radiation. Nateglinide Form H can be detected at concentrations at least about 2 percent by weight, and quantified at concentrations at least about 5 percent by weight.

The following examples are illustrative of certain aspects of the invention and are not intended to limit the scope of the invention, as claimed.

### EXAMPLE 1

Nateglinide Form B crystals were prepared by mixing 3 kg of nateglinide and 6 L of isopropanol, then stirring and heating the mixture to 40-45° C. until the nateglinide dissolved. A quantity of 0.3 kg activated carbon was added and stirring was continued for 15 minutes. The solution was filtered through a Nutsche pressure filter and added in the hot condition to 45 L of heptane that had been preheated to 42.5±2.5° C. With continuous mixing of the solution, 15 L of water, at a temperature of 42.5±2.5° C., were slowly added over a period of about 25 minutes and the mixture was allowed to cool; precipitation began as the temperature fell below about 39° C. The liquid was removed by centrifugation and the solids washed with 3 L of heptane, then centrifugation was continued to dry the solids. The dried solid nateglinide Form B was de-lumped using an oscillating granulator, then air jet milled to produce fine particles of nateglinide Form B, in which nateglinide Form H was not detected.

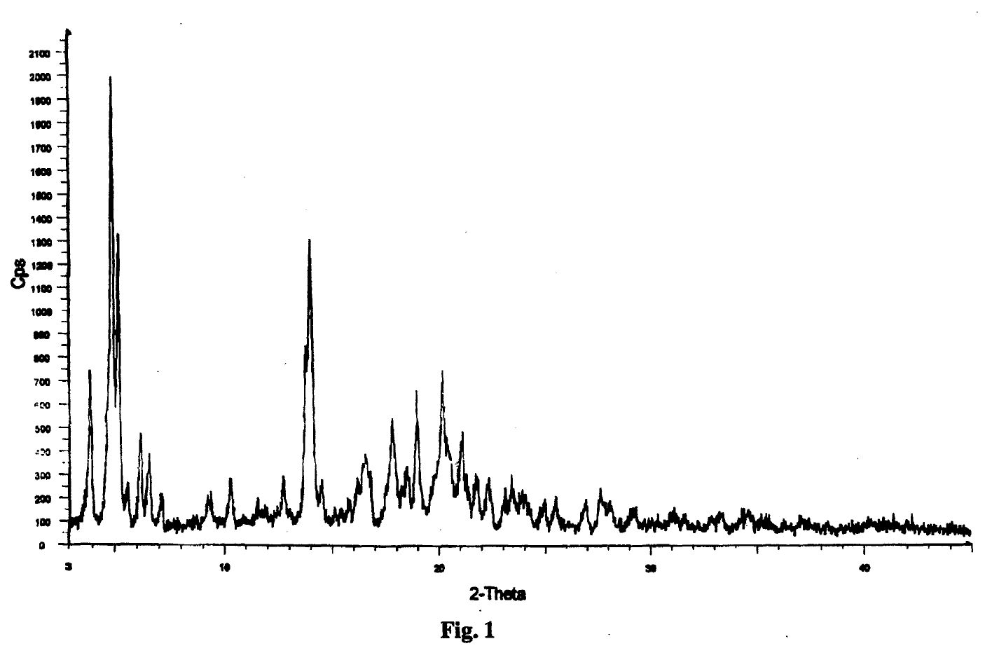

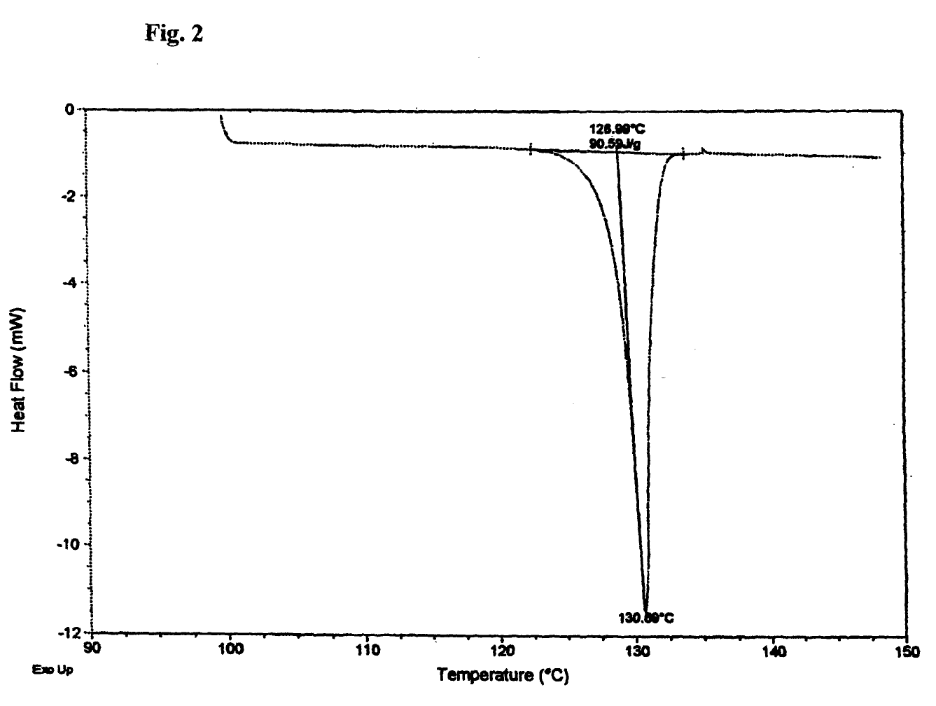

FIG. 1 is an X-ray diffraction pattern of nateglinide Form B that was prepared by this example; the x-axis is the 20θ angle, in degrees, and the y-axis is intensity. FIG. 2 is a differential scanning calorimetry curve of the nateglinide Form B prepared according to this example.

### EXAMPLE 2

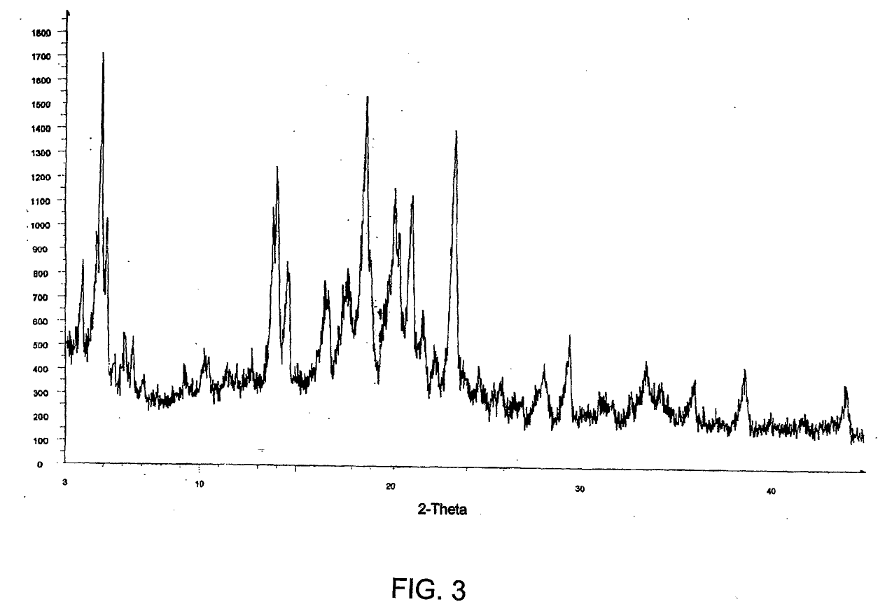

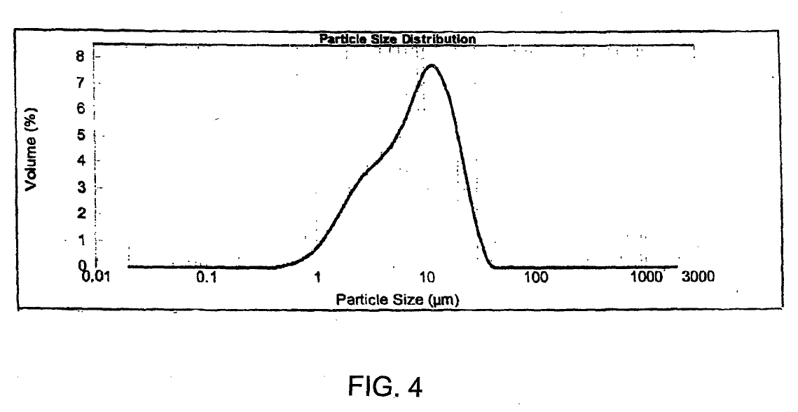

A mixture of nateglinide Form B, starch, and mannitol was prepared by charging 45 L of heptane into a reactor and chilling to 0-5° C. The starch (1.5 Kg) and mannitol (2 Kg) were added to the heptane and the mixture was stirred for about 15 minutes at 0 to 5° C. 3 kg of nateglinide Form B crystals were added to the mixture. The resulting suspension was stirred for about an hour at 0-5° C. and then centrifuged to remove liquid. The wet solid was dried in an air tray drier at about 90 to 100° C. for about 8 hours, until the loss on drying was less than about 0.5 percent by weight. The dried mixture was de-lumped, multi-milled and then air jet milled to produce fine particles having the size distribution: D₉₀≦20 μm; D₅₀≦10 μm; and D₁₀≦5 μm; as shown in FIG. 4. The term “D₉₀≦20 μm” means that 90 percent of the particles have a diameter not exceeding about 20 μm. The mixture obtained by the above procedure was free of nateglinde Form H crystals, as shown in the X-ray diffraction pattern of FIG. 3 where the x-axis is the 2θ angle, in degrees, and the y-axis is intensity.

### EXAMPLE 3

450 mL of heptane were charged into a round-bottom flask and chilled to 0-5° C. 15 g of starch and 20 g of mannitol were then added, and the mixture was stirred for about 15 minutes while maintaining the low temperature. 30 g of nateglinide Form B were added and the mixture was stirred for about an hour, without allowing the temperature to increase. Solids were isolated by filtration and then were dried for about 30 minutes in a fluid bed drier having an air inlet temperature of 40-50° C. and an air flow rate about 16 L/sec. The dried mixture was de-lumped and then air jet milled to the following particle size distribution: D₉₀≦20 μm; D₅₀≦10 μm; and D₁₀≦5 μm. The fine product was found to be free of nateglinide Form H crystals.

1. A process for preparing nateglinide Form B crystals, comprising:
a. dissolving nateglinide in a solvent;
b. adding the nateglinide solution, at temperatures about 40° C. to about 45° C., to a hydrocarbon liquid at temperatures about 40° C. to about 45° C.;
c. adding water and allowing the mixture to cool to form a solid; and
d. recovering solid nateglinide Form B.

2. The process of claim 1, wherein the solvent comprises an alcohol having 1 to about 4 carbon atoms, either branched or unbranched, or a ketone formed by one or two alkyl groups independently having 1 to about 4 carbon atoms, either branched or unbranched.

3. The process of claim 1, wherein the hydrocarbon liquid has about 4 to about 8 carbon atoms, either branched or unbranched.

4. The process of claim 1, wherein an amount of hydrocarbon liquid is about 5 to about 10 times the volume of solvent for the nateglinide.

5. The process of claim 1, wherein an amount of water is about 2 to about 4 times the volume of solvent for the nateglinide.

6. A process for preparing a mixture of nateglinide Form B and a pharmaceutical excipient, comprising:
a. mixing a pharmaceutical excipient and an organic liquid;
b. adding nateglinide Form B to the mixture; and
c. isolating a solid mixture of nateglinide Form B and excipient.

7. The process of claim 6, wherein the organic liquid is cooled to temperatures below ambient, prior to adding excipient.

8. The process of claim 6, wherein the organic liquid is cooled to less than about 10° C., prior to adding excipient.

9. The process of claim 6, wherein the pharmaceutical excipient comprises a mixture of two or more substances.

10. The process of claim 6, wherein the excipient comprises: a starch or starch derivative; a sugar; a cellulose or cellulose derivative; an alkaline earth metal salt; an aluminosilicate; an inorganic oxide; or a mixture of any two or more of the foregoing.

11. The process of claim 6, wherein the organic liquid comprises a hydrocarbon or an ether.

12. The process of claim 6, wherein the weight ratio of excipient to nateglinide Form B is about 0.2 to about 5.

13. The process of claim 6, wherein the weight ratio of excipient to nateglinide Form B is about 1.

14. A process for preparing a mixture of nateglinide Form B and a pharmaceutical excipient, comprising:
a. dissolving nateglinide in a solvent;
b. adding the nateglinide solution, at temperatures about 40° C. to about 45° C., to a hydrocarbon liquid at temperatures about 40° C. to about 45° C.;
c. adding water and allowing the mixture to cool to form a solid;
d. recovering solid nateglinide Form B;
e. adding the nateglinide Form B to a mixture of a pharmaceutical excipient and an organic liquid; and
f. isolating a solid mixture of nateglinide Form B and excipient.

15. The process of claim 6, wherein a pharmaceutical excipient comprises mannitol.

16. The process of claim 6, wherein a pharmaceutical excipient comprises a starch.

17. The process of claim 14, wherein the excipient comprises: a starch or starch derivative; a sugar; a cellulose or cellulose derivative; an alkaline earth metal salt; an aluminosilicate; an inorganic oxide; or a mixture of any two or more of the foregoing.

18. The process of claim 14, wherein the organic liquid comprises a hydrocarbon or an ether.

19. The process of claim 14, wherein a pharmaceutical excipient comprises mannitol.

20. The process of claim 14, wherein a pharmaceutical excipient comprises a starch.
